SEQUENCE LISTING (12 sequences)

SEQ ID NO: 1 - DNA, 20 bp, artificial
  VPS35 forward primer sequence
cgagaagacctcccgaatct

SEQ ID NO: 2 - DNA, 20 bp, artificial
  VPS35 reverse primer sequence
tccggagtgctgggtaaaac

SEQ ID NO: 3 - DNA, 20 bp, artificial
  beta-actin forward primer sequence
gatcattgctcctcctgagc

SEQ ID NO: 4 - DNA, 20 bp, artificial
  beta-actin reverse primer sequence
gtcaccttcaccgttccagt

SEQ ID NO: 5 - DNA, 21 bp, artificial
  VPS35-1 sense sequence
guggcagaucucuacgaactt

SEQ ID NO: 6 - DNA, 21 bp, artificial
  VPS35-1 antisense sequence
guucguagagaucugccactt

SEQ ID NO: 7 - DNA, 21 bp, artificial
  VPS35-2 sense sequence
gcacagcuagcugccaucatt

SEQ ID NO: 8 - DNA, 21 bp, artificial
  VPS35-2 antisense sequence
ugauggcagcuagcugugctt

SEQ ID NO: 9 - DNA, 21 bp, artificial
  control-1 sense sequence
uucuccgaacgugucacgutt

SEQ ID NO: 10 - DNA, 21 bp, artificial
  control-1 antisense sequence
acgugacacguucggagaatt

SEQ ID NO: 11 - DNA, 21 bp, artificial
  control-2 sense sequence
gagauagggugucucgcuctt

SEQ ID NO: 12 - DNA, 21 bp, artificial
  control-2 antisense sequence
gagcgagacacccuaucuctt

(19) United States
(12) Patent Application Publication
(10) Pub. No.: US 2008/0214482 A1
(43) Pub. Date: Sep. 4, 2008

(54) Retromer-based assays and methods for treating alzheimer's disease

(76) Inventors: Scott Small, Millerton, NY (US); Tae-Wan Kim, East Brunswick, NJ (US)

Correspondence Address: COOPER & DUNHAM, LLP, 1185 AVENUE OF THE AMERICAS, NEW YORK, NY, 10036, US

(21) Appl. No.: 11/599,896

(22) Filed: Nov. 14, 2006

Related U.S. Application Data
(60) Provisional application No. 60/737,531, filed on Nov. 15, 2005.

(51) Int. Cl. A61K 31/7088; G01N 33/68; C12Q 1/68; C12N 5/06; A61P 25/28

(52) U.S. Cl. 514/44; 435/7.8; 435/6; 435/7.1; 435/375

(57) ABSTRACT

This invention provides a method for determining whether an agent causes an increase in the expression of a retromer complex protein. This invention further provides a method for determining whether an agent causes an increase in the activity of a retromer complex. This invention also provides a method for increasing the expression of a retromer complex protein in a cell. This invention provides a method for treating a subject afflicted with Alzheimer's disease. This invention further provides a pharmaceutical composition as well as an article of manufacture.

This application claims the benefit of U.S. Provisional Application No. 60/737,531, filed Nov. 15, 2005, the contents of which are incorporated herein by reference into the subject application.

This invention was made with support under United States Government Grant Nos. AG08702 and AG00949 from the National Institutes of Health. Accordingly, the United States Government has certain rights in the subject invention.

Throughout this application, certain publications are referenced. Full citations for these publications, as well as additional related references, may be found immediately preceding the claims. The disclosures of these publications are hereby incorporated by reference into this application in order to more fully describe the state of the art as of the date of the invention described and claimed herein.

### BACKGROUND OF THE INVENTION

A range of studies have established that elevated concentrations of Aβ peptide, a cleaved product of amyloid precursor-protein (APP), is fundamental to AD pathogenesis¹. In the rare autosomal-dominant form of Alzheimer's disease (AD) molecular defects in APP itself or in components of the γ-secretase result in increased Aβ production². These defects, however, do not exist in the late-onset form of AD, accounting for 95% of all cases, and the factors that cause increased Aβ concentrations in sporadic AD—by increasing production or decreasing clearance—remains undetermined.

In principle, profiling patterns of gene expression using techniques like microarray is a powerful approach for isolating molecular differences between healthy and diseased tissue^{3, 4}. In practice, however, these techniques suffer a number of analytic challenges, particularly when applied to disorders of the brain^{3, 5}.

Mapping the precise spatiotemporal profile of AD can be used to address some of the analytic challenges inherent to microarray⁵. By identifying the brain site most vulnerable to AD, microarray data can be generated selectively from this region, maximizing expression differences between AD and controls thereby enhancing signal amplitude. At the same time, by identifying a neighboring brain region relatively resistant to AD, microarray data from this region can be used to normalize against inter-individual sources of global variance, thereby constraining signal noise.

Cognitive studies have identified the hippocampal formation as a gross anatomical structure particularly vulnerable to AD^{6, 7}. The hippocampus itself, however, is made up of anatomically distinct subregions, and recent microarray studies have shown that each hippocampal subregion expresses a unique molecular profile^{8, 9}. Although all subregions ultimately manifest AD pathology, these molecular observations underlie the assumption that AD targets the hippocampus with regional selectivity¹⁰. In vitro markers of AD pathology—such as amyloid plaques, neurofibrillary tangles, or cell loss—applied to post-mortem tissue have confirmed this assumption. Most post-mortem studies have suggested that either the entorhinal cortex¹¹⁻¹⁶ or the CA1 subfield^{11-13, 17-19} are candidate sites of primary vulnerability, although some studies have implicated other subregions as we11²⁰. Indeed, based on these findings the CA1 subfield has been chosen as the target brain region in previous microarray studies investigating expression profiles in the hippocampus of AD brains²¹⁻²³. In many post-mortem studies, the entorhinal cortex and the CA1 subfield were not assessed simultaneously, accounting in part for the reported inconsistencies in determining which subregion is most vulnerable to AD. More generally, however, isolating the hippocampal subregion most vulnerable to AD may be challenging relying on post-mortem studies alone. Not only are post-mortem studies biased against the earliest and most discriminatory stages of disease, but synaptic dysfunction is an early defect that can, in principle, occur independent of amyloid plaques, neurofibrillary tangles, and cell loss²⁴.

With these considerations in mind, variants of fMRI (functional magnetic resonance imaging) have been developed that are sensitive to synaptic dysfunction²⁵ and that can visualize individual hippocampal subregions in living subjects²⁵⁻²⁷. These techniques have been used to image AD patients with frank dementia²⁶ and healthy subjects suspected of harboring the earliest stages of AD dysfunction²⁷. Although both hippocampal subregions were implicated in these studies, the entorhinal cortex, not the CA1 subfield, was found to be the single hippocampal subregion most vulnerable to AD. Based on these imaging findings it was decided to focus on the entorhinal cortex, not the CA1 subfield, as the target brain region in performing our microarray analysis of AD. In terms of identifying a brain region relatively resistance to AD, imaging findings in living subjects²⁷ agree with almost all post-mortem studies¹¹⁻¹⁹ showing that the dentate gyrus is the neighboring hippocampal subregion most resistant to AD. Finally, beyond contributing to the spatial pattern of AD, imaging studies have also informed on its temporal profile. Notably, the difference in entorhinal function between controls and affected individuals has been shown to be age-independent²⁶⁻²⁸, implying that once a pathogenic molecule is altered from baseline it does not change with age²⁶⁻²⁸.

### SUMMARY OF THE INVENTION

This invention provides a method for determining whether an agent causes an increase in the expression of a retromer complex protein, comprising the steps of (a) contacting the agent with a eukaryotic cell under conditions which, in the absence of the agent, permit expression of the retromer complex protein, (b) after a suitable period of time, determining the amount of expression in the cell of the retromer complex protein; and (c) comparing the amount of expression determined in step (b) with the amount of expression which occurs in the absence of the agent, whereby an increased amount of expression in the presence of the agent indicates that the agent causes an increase in the expression of the retromer complex protein.

This invention also provides a method for determining whether an agent causes an increase in the activity of a retromer complex, comprising the steps of (a) contacting the agent with a eukaryotic cell under conditions which, in the absence of the agent, permit activity of the retromer complex, (b) determining the amount of activity in the cell of the retromer complex, and (c) comparing the amount of activity determined in step (b) with the amount of activity which occurs in the absence of the agent, whereby an increased amount of activity in the presence of the agent indicates that the agent causes an increase in the activity of the retromer complex.

This invention also provides a method for increasing the expression of a retromer complex protein in a cell comprising introducing into the cell an agent which specifically increases the expression of the retromer complex protein in the cell.

This invention further provides a method for treating a subject afflicted with Alzheimer's disease comprising administering to the subject a therapeutically effective amount of an agent which specifically increases the expression of the retromer complex protein in the cells of the subject's brain which express AP peptide.

This invention further provides a pharmaceutical composition comprising (a) an agent which specifically increases the expression of a retromer complex protein when introduced into a cell; and (b) a pharmaceutically acceptable carrier.

Finally, this invention provides an article of manufacture comprising (a) a packaging material having therein an agent which specifically increases the expression of a retromer complex protein when introduced into a cell; and (b) a label indicating a use for the agent in treating a subject afflicted with Alzheimer's disease.

### BRIEF DESCRIPTION OF THE FIGURES

FIGS. 1A-B. A spatiotemporal model of Alzheimer's disease used to guide microarray acquisition and analysis.

A. The spatial component of the model. The hippocampal formation is made up of separate subregions, the entorhinal cortex (EC), the subiculum (Sub), the CA1 and CA3 subfields, and the dentate gyrus (DG). Prior histological and imaging studies have established that the entorhinal cortex is the hippocampal subregion most vulnerable, and that the dentate gyrus is relatively resistant, to Alzheimer's disease. Comparing gene-expression profiles from the entorhinal cortex of patients and controls maximizes the detection of subtle but relevant expression differences. Gene-expression profiles from the dentate gyrus can be used to minimize global sources of variance.

B. The temporal component of the model. Imaging entorhinal cortex across different ages and over time has established that the difference in function between Alzheimer's disease (black circles) and controls (grey circles) is age-independent. The absence of a group-by-age interaction can be used as an analytic filter against false-positive findings. In principle, as shown, a molecule related to Alzheimer's disease can be higher or lower than controls.

FIGS. 2A-C. mRNA levels of VPS35, a retromer trafficking molecule, best conformed to the spatiotemporal model of Alzheimer's disease.

A. Normalized entorhinal cortex expression (EC/DG=entorhinal cortex expression divided by dentate gyrus expression) is shown individually for 6 control and 6 Alzheimer's disease (AD) cases (upper graph). Analyzing the data region-by-region (lower graph) shows that the effect is driven by a difference in the entorhinal cortex.

B. VPS35 conformed to the temporal component of the model. Normalized expression levels from the entorhinal cortex are shown for each control (grey circles) and AD (black circle) case across the age-range. As shown, the difference in VPS35 expression between AD and controls is age-independent.

C. Differential expression of VPS35 is confirmed with RT-PCR in the original sample upper graph) and in an independent sample (lower graph).

FIGS. 3A-B. Protein levels of VPS35 and VPS26 are differentially reduced in Alzheimer's disease.

A. VPS35 protein is differentially reduced in Alzheimer's disease. Normalized entorhinal cortex expression, as determined by quantitative Western blotting, is shown individually for 9 control and 12 Alzheimer's disease (AD) cases (upper graph). Analyzing the data region-by-region (middle graph) shows that the effect is driven by a difference in the entorhinal cortex. Immunocytochemical staining (lower graph) of the entorhinal cortex isolated from an Alzheimer's disease case shows that VPS35 protein is expressed predominantly in the pyramidal cells (bar=100 um).

B. VPS26, a second retromer protein, is also differentially reduced in Alzheimer's disease. Normalized entorhinal cortex expression, as determined by quantitative Western blotting, is shown individually for 5 control and 10 Alzheimer's disease (AD) cases (upper graph). Analyzing the data region-by-region (middle graph) shows that the effect is driven by a difference in the entorhinal cortex. VPS35 and VPS26 are significantly correlated with each other (lower graph).

FIGS. 4A-B. VPS35 regulates Aβ levels.

A. Lowering VPS35 protein increases Aβ levels. Two Western blot examples (left panel) show that siRNA directed against VPS35 reduces protein level by approximately 35%, compared to non-silencing control. Actin levels were unaffected by siRNA. A 35% reduction in VPS35 levels led to a 37% increase in endogenous Aβ production (right panel).

B. Elevating VPS35 protein decreases Aβ levels. Three Western blot examples (left panel) show that stably transfecting VPS35 using a cDNA vector pEF6-V5 increases VPS35 levels compared to the empty vector alone. Actin levels were unaffected by transfection. Increasing VPS35 levels led to a 40% decrease in endogenous AP production (right panel).

FIGS. 5A-B. Proposed model for retromer dysfunction and Aβ processing.

A. Normal retromer function. VPS35 is the core component of the retromer trafficking complex (box). The retromer traffics type-I membrane proteins (bars) as its cargo from the endosome to the trans-golgi network (TGN). Although Aβ is produced in multiple organelles, β-site APP-cleaving enzyme (BACE) activity is maximized in the acidic environment of the endosome.

B. Retromer dysfunction. As previously established, a reduction in VPS35 and/or VPS26 causes retromer dysfunction (box), back-logging retromer cargo (bars) in the endosome and the cell surface. Retromer dysfunction in predicted to increase the concentration of BACE or APP in the endosome, directly or indirectly via SorLA or other VPS10-containing proteins (bars), leading to increased Aβ production.

### DETAILED DESCRIPTION OF THE INVENTION

Terms

As used in this application, except as otherwise expressly provided herein, each of the following terms shall have the meaning set forth below.

As used herein, “administering” an agent can be effected or performed using any of the various methods and delivery systems known to those skilled in the art. The administering can be performed, for example, intravenously, via cerebrospinal fluid, orally, nasally, via implant, transmucosally, transdermally, intramuscularly, and subcutaneously.

As used herein, “agent” shall mean any chemical entity, including, without limitation, a protein, an antibody, a nucleic acid, a small molecule, and any combination thereof.

As used herein, “antibody” shall include, by way of example, both naturally occurring and non-naturally occurring antibodies. Specifically, this term includes polyclonal and monoclonal antibodies, and antigen-binding fragments (e.g., Fab fragments) thereof. Furthermore, this term includes chimeric arntibodies (e.g., humanized antibodies) and wholly synthetic antibodies, and antigen-binding fragments thereof.

As used herein, “microarray” shall mean (a) a solid support having one or more compounds affixed to its surface at discrete loci, or (b) a plurality of solid supports, each support having one or a plurality of compounds affixed to its surface at discrete loci. The instant microarrays can contain all possible permutations of compounds within the parameters of this invention. For example, the instant microarray can be a disease-specific microarray, a species-specific microarray, or a tissue-specific microarray.

As used herein, “pharmaceutically acceptable carrier” shall mean any of the various carriers known to those skilled in the art.

The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

As used herein, “retromer complex” shall mean a complex of proteins, wherein this complex (a) comprises a single VPS35 protein and one or more other proteins, and (b) performs functions including, for example, trafficking type I membrane proteins and acting to increase the concentration of membrane proteins in the endosome and trans-golgi network. “Retromer complex protein” shall mean one of the proteins contained within a retromer complex.

As used herein, “subject” shall mean any animal, such as a human, non-human primate, mouse, rat, guinea pig or rabbit.

As used herein, “suitable period of time” shall mean an amount of time sufficient to permit expression of the retromer complex protein.

As used herein, “therapeutically effective amount” means an amount sufficient to treat a subject afflicted with a disease (e.g. Alzheimer's disease) or a complication associated with a disease.

As used herein, “treating” shall mean slowing, stopping or reversing the progression of a disease (e.g. Alzheimer's disease).

### EMBODIMENTS OF THE INVENTION

This invention provides a method for determining whether an agent causes an increase in the expression of a retromer complex protein, comprising the steps of (a) contacting the agent with a eukaryotic cell under conditions which, in the absence of the agent, permit expression of the retromer complex protein; (b) after a suitable period of time, determining the amount of expression in the cell of the retromer complex protein; and (c) comparing the amount of expression determined in step (b) with the amount of expression which occurs in the absence of the agent, whereby an increased amount of expression in the presence of the agent indicates that the agent causes an increase in the expression of the retromer complex protein.

In one embodiment of the above method, the retromer complex protein is VPS35. In another embodiment, the retromer complex protein is selected from the group consisting of VPS17, VPS26, VPS29, SorLa, sorting nexin 1 and sorting nexin 2. In a further embodiment, the cell is present in a cell culture. The cell may be a brain cell. In another embodiment of the above method, determining the amount of expression is performed by determining the amount of retromer complex protein-encoding mRNA in the cell. In yet another embodiment, determining the amount of expression is performed by determining the amount of retromer complex protein in the cell. In a further embodiment, determining the amount of retromer complex protein in the cell is performed using an antibody specific for such protein.

This invention also providers a method for determining whether an agent causes an increase in the activity of a retromer complex, comprising the steps of (a) contacting the agent with a eukaryotic cell under conditions which, in the absence of the agent, permit activity of the retromer complex; determining the amount of activity in the cell of the retromer complex; and (c) comparing the amount of activity determined in step (b) with the amount of activity which occurs in the absence of the agent, whereby an increased amount of activity in the presence of the agent indicates that the agent causes an increase in the activity of the retromer complex.

In one embodiment, the amount of activity of the retromer complex is determined by measuring the amount of trafficking of type I membrane proteins in a cell. In another embodiment, the amount of activity of the retromer complex is determined by measuring the amount by which the concentration of membrane proteins are concentrated in the endosome and/or trans-golgi network.

In one embodiment of the above method, the retromer complex protein is VPS35. In another embodiment, the retromer complex protein is selected from the group consisting of VPS17, VPS26, VPS29, SorLa, sorting nexin 1 and sorting nexin 2. In a further embodiment, the cell is present in a cell culture. The cell may be a brain cell.

This invention further provides a method for increasing the expression of a retromer complex protein in a cell comprising introducing into the cell an agent which specifically increases the expression of the retromer complex protein in the cell.

In one embodiment of the above method, the retromer complex protein is VPS35. In another embodiment, the retromer complex protein is selected from the group consisting of VPS17, VPS26, VPS29, SorLa, sorting nexin 1 and sorting nexin 2. In a further embodiment, the cell is present in a cell culture. The cell may be a brain cell. In yet another embodiment, the agent is a nucleic acid. The nucleic acid may be, for example, an expression vector encoding one or more retromer complex proteins. In the preferred embodiment, the nucleic acid is an expression vector encoding VPS35.

This invention also provides a method for treating a subject afflicted with Alzheimer's disease comprising administering to the subject a therapeutically effective amount of an agent which specifically increases the expression of the retromer complex protein in the cells of the subject's brain which express Aβ peptide.

In one embodiment of the above method, the retromer complex protein is VPS35. In another embodiment, the retromer complex protein is selected from the group consisting of VPS17, VPS26, VPS29, SorLa, sorting nexin 1 and sorting nexin 2. In a further embodiment, the agent is a nucleic acid. The nucleic acid may be, for example, an expression vector encoding one or more retromer complex proteins. In the preferred embodiment, the nucleic acid is an expression vector encoding VPS35.

This invention also provides a pharmaceutical composition comprising an agent which specifically increases the expression of a retromer complex protein when introduced into a cell; and a pharmaceutically acceptable carrier.

Finally, this invention provides an article of manufacture comprising a packaging material having therein an agent which specifically increases the expression of a retromer complex protein when introduced into a cell; and a label indicating a use for the agent in treating a subject afflicted with Alzheimer's disease.

This invention is illustrated in the Experimental Details section which follows. This section is set forth to aid in an understanding of the invention but is not intended to, and should not be construed to limit in any way the invention as set forth in the claims which follow thereafter.

### EXPERIMENTAL DETAILS

### Synopsis

Although, in principle, gene-expression profiling is well suited to isolate pathogenic molecules associated with Alzheimer's disease, techniques like microarray present unique analytic challenges when applied to disorders of the brain. These challenges are addressed here by first constructing a spatiotemporal model, predicting a priori how a molecule underlying AD should behave anatomically and over time. Then, guided by the model, gene-expression profiles of the entorhinal cortex and the dentate gyrus, harvested from the brains of AD cases and controls covering a broad age-span were generated. Among many expression differences, the retromer trafficking molecule VPS35 best conformed to the spatiotemporal model of AD. Western blotting confirmed the abnormality, establishing that VPS35 levels are reduced in brain regions selectively vulnerable to AD. VPS35 is the core molecule of the retromer trafficking complex and further analysis revealed that VPS26, another member of the complex, is also downregulated in AD. Cell culture studies, using siRNA or expression vectors, showed that VPS35 regulates AP peptide levels, establishing the relevance of the retromer complex to AD. Reviewing these findings in the context of recent studies suggests how downregulation of the retromer complex in AD can regulate local levels of AP peptide.

The spatial profile of AD dysfunction can be used to enhance microarray signal-to-noise, while the temporal profile of AD can be used to filter false-positive findings. Employing this analytic approach, our microarray analysis identified an AD-related defect in the retromer trafficking molecule VPS35.

Because of the complex and often discordant relationship between mRNA and protein level²⁹⁻³⁵, Western blot analysis was used to confirm the abnormality in VPS35 and to establish that VPS35 protein levels are abnormally low in AD. Finally, cell-culture studies were used to establish a relationship between VPS35 and Aβ peptide, thus confirming the relevance of this trafficking complex to AD pathophysiology.

### Materials and Methods

Human Brain Samples: Alzheimer's disease (AD) and control brain samples were obtained at autopsy under a protocol approved by the institution's review board. The entorhinal cortex and the dentate gyrus were identified and sectioned using strict anatomical criteria following New York Brain Bank procedures, Subregion dissection was performed in the fresh state and then samples were snap frozen in liquid nitrogen and stored at −80° C.

Gene-expression profiling: Six brains with pathologically proven AD and from 6 brains free of pathology, purposely selected from subjects that cover a broad age-span (33-98 years of age). For each of the 12 brains, total RNA was extracted from entorhinal cortex and dentate gyrus tissue with TRIzol reagent (Invitrogen, Carlsbad, Calif.) and was purified with RNeasy column (Invitrogen). 10 μg total RNA were used to prepare double-stranded cDNA (Superscript, Invitrogen). The T7-(dT)₂₄ primer for cDNA synthesis contained a T7 RNA polymerase promoter site. An in vitro transcription reaction with biotin-labeled ribonucleotides was performed on the cDNA to produce cRNA probes (Bioarray High Yield RNA Transcript Labeling Kit, ENZO Life Sciences, Farmingdale, N.Y.). In the Gene Chip Facility of Columbia University, HG-U133A microarrays (GeneChip, Affymetrix, Santa Clara, Calif.) were hybridized with fragmented cRNA for 16 h in a 45° C. incubator with constant rotation at 60 g. Microarrays were washed and stained on a fluidics station, and scanned using a laser confocal microscope. HG-U133A microarrays were analyzed with Affymetrix Microarray Suite v5.0 and GeneSpring v5.0.3 (Silicon Genetics, Redwood City, Calif.) software, and scaled to a value of 500. Samples which had a 3′/5′ ratio of control genes actin and GAPDH greater than 7, were excluded from analysis. Transcripts whose detection levels had a p-value greater than 0.05 were excluded and raw data of the 7610 included molecules can be found as online supplementary material.

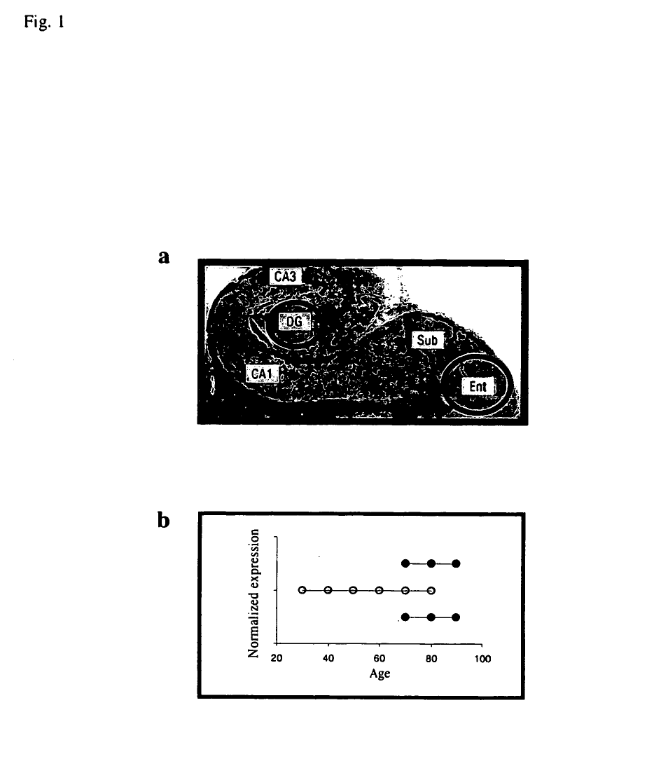

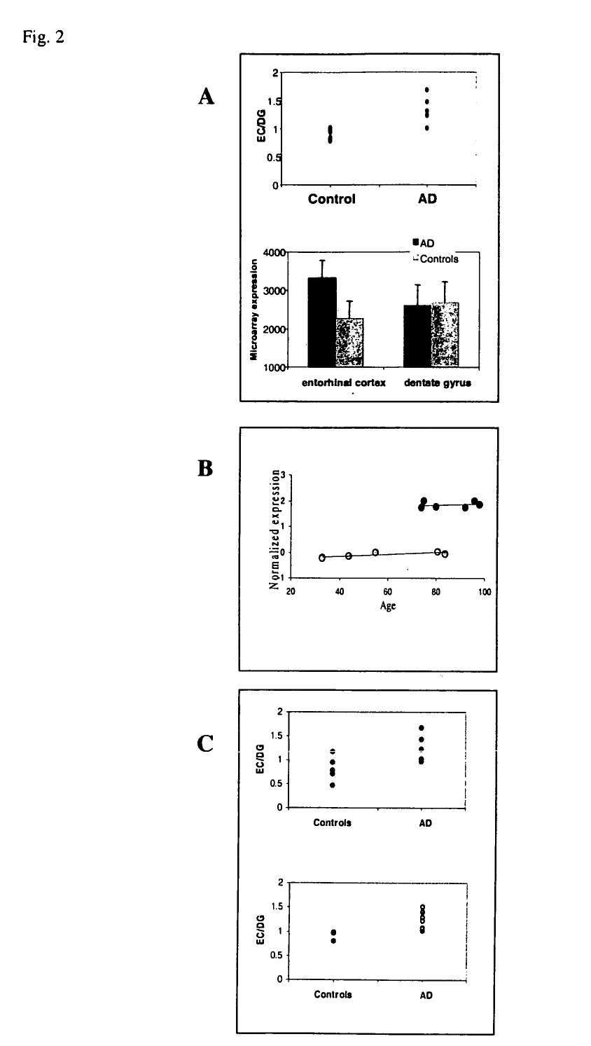

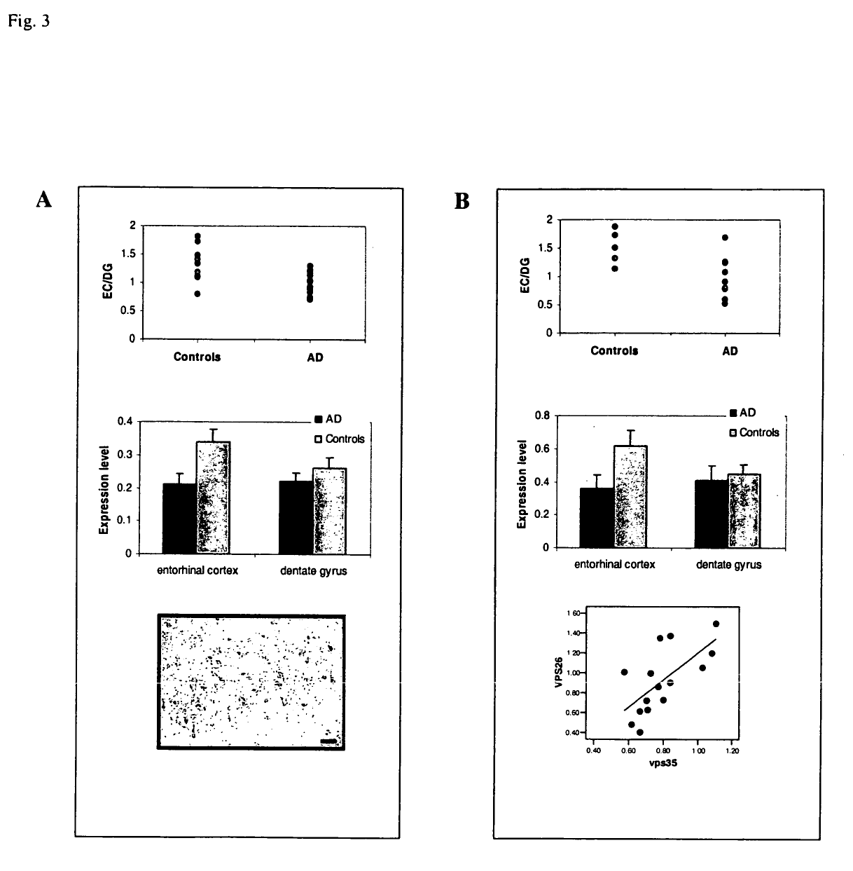

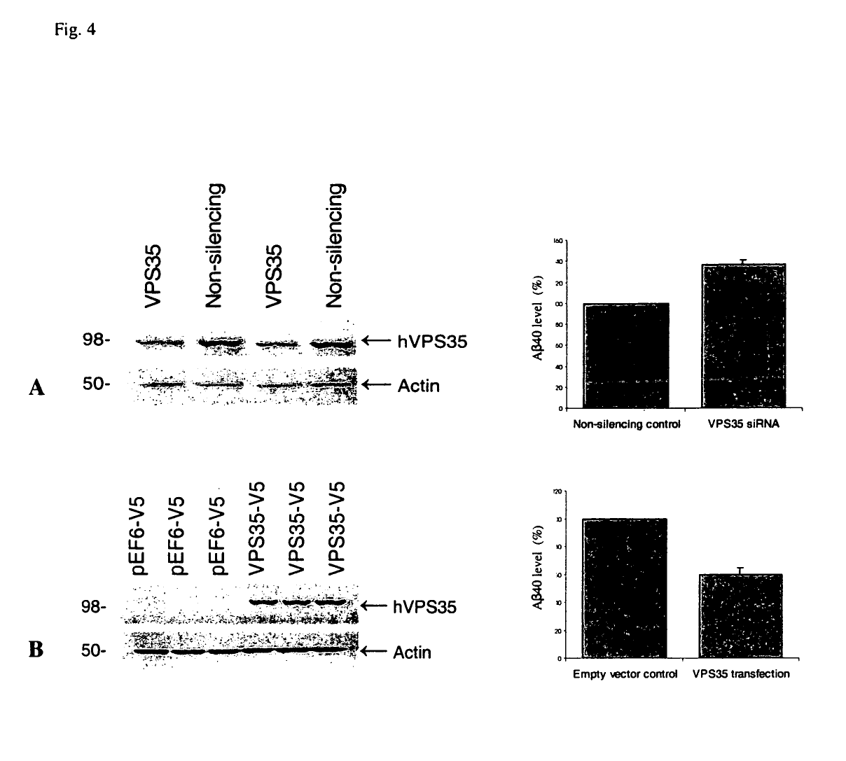

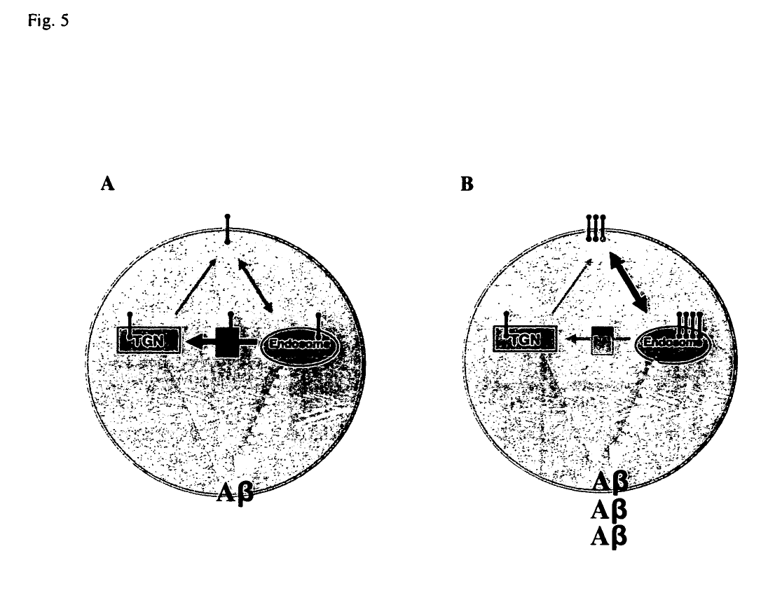

Microarray data analysis: Based on the spatial component of the model, pathogenic molecules—those underlying AD—should be differentially expressed in the entorhinal cortex compared to the dentate gyrus (FIG. 1a). According to the temporal component of the model, the expression differences between AD and controls should be age-independent (FIG. 1b).

In accordance with this model, statistical analysis was performed in two steps. First, the expression levels of each molecule measured in the entorhinal cortex was divided by expression levels of the same molecule measured in the dentate gyrus of the same individual. This ratio is performed to normalize entorhinal cortex expression levels against global sources of inter-individual variance—such as environmental differences during life and the dying process. An ANOVA was then performed where group was included as the fixed factor and the normalized expression levels (EC/DC,) were included as dependent variables, and age was included as a covariate. Because of the concern that a significant difference in dentate gyrus expression, not expression differences in entorhinal cortex, might underlie a significant difference in the ratios, a secondary analysis was performed on those molecules whose ratios were significantly different between groups. A repeated-measures ANOVA was used, where expression levels of each region (entorhinal cortex vs. dentate gyrus) was included as the within-subject variables, and group (AD vs. control) was included as the between-subject variables, and age was included as a covariate. This analysis allows the expression levels from each hippocampal subregion to be examined individually.

Next, it was determined which among the molecules that conformed to the spatial pattern, also conformed to the temporal component of the model. The same ANOVA was repeated but in this case included an age-by-group factor as an additional covariate. In doing so, only molecules that conform to the temporal component will yield a significant effect.

Real-time quantitative PCR: 2 μg total RNA and oligo(dT)₁₂₋₁₈ primer were used to generate single-stranded cDNA (Superscript, Invitrogen). The relative amount of VPS35 and □-Actin mRNAs were measured by real-time quantitative PCR using SmartCycler II (Cepheid, Sunnyvale, Calif.). The specific primer sets used were: VPS35 forward, 5′-CGAGAAGACCTCCCGAATCT-3′; VPS35 reverse, 5′-TCCGGAGTGCTGGGTAAAAC-3′; β-ACTIN forward, 5′-GATCATTGCTCCTCCTGAGC-3′; β-ACTIN reverse, 5′-GTCACCTTCACCGTTCCAGT-3′. The 25 μl reaction mixture was prepared following manufacturer's suggestion using 1 puReTaq Ready-To-Go PCR bead (Amersham, Amersham, UK), 50 mM MgCl₂, 1:10,000 SYBR Green (Molecular Probes, Eugene, Oreg.), 25 μM of each primer, and 800 ng cDNA. Following 60 s at 95° C., 40 cycles of 10 s at 95° C., 30 s at 66° C., 30 s at 72° C., and 20 s at 86° C. (80° C. for β-Actin reaction) were carried out. β-Actin expression was used for normalization.

Antibody development: anti-VPS35 antibody was developed in house. Full-length cDNA clones encoding human VPS35p were acquired from the integrated molecular analysis of genomes and their expression (I.M.A.G.E.) clone collection. The coding sequences were amplified using PCR and sub-cloned into mammalian expression plasmids with or without the V5 epitope tag. Two different rabbit polyclonal antibodies raised against the synthetic peptides corresponding to the 15 C-terminal amino acids of human VPS35p (hVPS35p) or against full-length hVPS35p GST fusion proteins were developed. These antibodies selectively recognized hVPS35p in immunoprecipitation and Western blot analyses. Anti-VPS26p antibody was purchased commercially from Novus Biological (Littleton, Colo.).

Western Blotting: Frozen human hippocampal sections of dentate gyrus and entorhinal cortex are soaked in 5 volumes of solution (0.32M Sucrose, 0.5 mM CaCl₂, 1 mM MgCl₂, 1 mM NaHCO₃) supplemented with protease Inhibitor cocktail (Roche; Nutley, N.J.) for 15-30 minutes. Samples were homogenized on ice with 12 strokes at 900 rpm using a motor-operated Tephlon-pestle homogenizer. Homogenate was centrifuged at 240×g for 10 min at 4° C., and the supernatant is saved (S1). Western blotting was performed on 3-20 μg of protein sample (S1). Blots were incubated sequentially in TBS for 1 hour, the antibody of interest overnight and the appropriate fluorescently-labeled secondary antibody for 1 hour, and evaluated using the Odyssey Infrared Imaging System (LI-COR Biotechnology; Lincoln, Nebr.).

Human brain immunocytochemistry: Coronal blocks of human hippocampal formation were frozen-sectioned using a Microm cryostat at 8 μm thickness. Tissue was either directly quick-frozen or, in some cases, fixed 4% paraformaldehyde in PBS for 18 hr, cryoprotected in 25% sucrose in PBS, and then quick-frozen. Sections on slides were postfixed with 4% paraformaldehyde in PBS, washed with PBS, then treated with 3% H₂O₂, washed, and preincubated for 1 hr in Block solution consisting of 2% horse serum (Vector, Burlingame, Calif.), 1% bovine serum albumin (Sigma-Aldrich Chemical Co., St Louis, Mo.) and 0.1% Triton X-100 (Sigma) in PBS. Slides were then incubated 18 hr at 4° C. in diluted (1:500 in Block solution) polyclonal antiserum to VPS35p. After washing with PBS, immunoreactivity was detected by an avidin-biotin linked peroxidase method, using successive incubations and washes with goat anti-rabbit biotinylated IgG, Vectastain ABC-Elite reagent (Vector), and diaminobenzidine (Sigma) chromogen reagent. Sections were dehydrated and mounted using Permount (Fisher Scientific, Pittsburgh, Pa.).

RNA Interference and Delivery: Synthetic 21-23 mer small interfering RNAs (siRNAs) corresponding to human VPS35 were designed based on the published criteria³⁶ and synthesized by Qiagen, Inc. The following sequences were used for VPS35 siRNAs: 1) sense VPS35-1, 5′-GUGGCAGAUCUCUACGAAC dTdT; 2) antisense VPS35-1, 5′-GUUCGUAGAGAUCUGCCACdTdT; 3) sense VPS35-2, 5′-GCACAGCUAGCUGCCAUCAdTdT; 4) antisense VPS35-2, 5′-UGAUGGCAGCUAGCUGUGC dTdT. The following sequences were used for control siRNAs: 1) sense control-1, 5′-UUCUCCGAACGUGUCACGU dTdT; 2) antisense control-1, 5′-ACGUGACACGUUCGGAGAA dTdT; 3) sense control-2, 5′-GAGAUAGGGUGUCUCGCUC dTdT; 4) antisense control-2, 5′-GAGCGAGACACCCUAUCUC dTdT. Annealing for duplex siRNA was performed as described³⁷. Hela cells were maintained in DMEM supplemented with 10% fetal bovine serum and penicillin/streptomycin. Three hours post-transfection, cells were washed 3 times with ×PBS, and further transfected with 2 ug of each siRNA duplex using Oligofectamine™ (Invitrogen) according to the manufacturer's instruction. VPS35 cDNAs and Transfection: Full-length human VPS35 CDNA was amplified by PCR from the I.M.A.G.E. clone (#3162255) and subcloned into the expression vector pEF6-TOPO with the V5/His epitope at the C-terminus of VPS35 (VPS35-V5) . HEK293 cells were stably transfected with VPS35 expression constructs using Superfect (Quiagen) transfection reagent by manufacturer's protocol.

Aβ Analysis: 72-96 hours post-transfection, conditioned medium was collected, centrifuged at 15,000×g for 15 min at 4° C., and Sandwich ELISA was performed using Signal Select™ Human β-amyloid 1-40 and β-amyloid 1-42 ELISA Kits (Biosource International, Inc., Camarillo, Calif., USA) according to the manufacturer's protocol. Samples were measured in triplicate wells and each experiment was conducted three times.

### Results

The first analysis, testing for molecules that conformed to the spatial component of the model, revealed 33 molecules with at a p<0.01 (Table 1). Because of type-I error incurred by multiple comparisons³⁸ it is assumed that only a few of these molecules are true-positives, and the temporal component of the model was used to filter against false-positivity. Among the 33 transcripts, expression levels of 5 molecules conformed to the temporal component of the model: VPS35, Beclin 1, COP9 homolog, proteasome beta 4 subunit, and nucleobindin 2. Since VPS35 best conformed to the temporal model (F=14.7; p=0.005; FIG. 2b) a more detailed analysis of this molecule was pursued, although interest in other molecules has not been ruled-out. Secondary analysis using a repeated-measures ANOVA was performed to examine VPS35 expression levels region-by-region. A significant region X group interaction was observed (F=14. 9; p=0.003) and visual inspection of the data (FIG. 2a) shows that the effect is driven primarily by between-group differences in the entorhinal cortex and not the dentate gyrus.

RT-PCR was used to measure VP35 mRNA from all 24 tissue samples and an ANOVA confirmed the AD-related abnormality in normalized VPS35 levels (F=10.4; p=0.01) (FIG. 2c). This effect was then replicated in a second independent set of tissue samples (F=7.8; p=0.02) (FIG. 2c).

Because protein, not mRNA, is the functionally meaningful end-product of gene expression, a number of studies have explored the relationship between levels of mRNA and protein. Importantly, although positive correlations are observed, in some cases an inverse correlation between mRNA and protein is found, and often there is no correlation at all²⁹⁻³⁵. Thus, at best, mRNA studies can identify a molecule that is abnormally expressed, but cannot establish whether the protein product is in fact elevated or reduced. Western blotting is required, therefore, to first confirm that VPS35 protein levels are in fact abnormal is AD, and, if so, to determine the direction of the effect.

Western blot analysis was performed on the entorhinal cortex and the dentate gyrus harvested from 12 brains with AD and 9 controls, and the observed levels of VPS35 protein were normalized against actin. As with the mRNA, a similar ANOVA performed on the normalized protein levels, covarying for the group difference in age, revealed a relative decrease in the AD cases (F=8.7; p=0.008) (FIG. 3a). Here, again, the effect was found to be age-independent. Examining the protein data region-by-region demonstrates that the effect is driven by a difference in the entorhinal cortex (FIG. 3a). Thus, analysis at the protein level confirms that VPS35 is abnormal in AD. However, as observed in previous studies²⁹⁻³³, the direction of the effect is inversed. Specifically, the level of VPS35 protein is differentially reduced in the entorhinal cortex of AD brains (FIG. 3). This inverse relationship of high mRNA and low protein suggests either accelerated degradation of the VPS35 protein^{34, 35} or slower turnover the VPS35 mRNA. Immnuocytochemistry showed that VPS35 is predominately expressed in pyramidal neurons (FIG. 3a).

VPS35 is the core molecule of the retromer trafficking complex. Previous studies have shown that protein levels of VPS35 and VPS26, another key member of the retromer complex, are typically cross-correlated- where a reduction in one leads to a reduction in the other³⁹⁻⁴¹. The expression levels of VPS26 protein were therefore measured in a subset of the same tissue samples. Results revealed that like VPS35, VPS26 is also differentially reduced in the entorhinal cortex of AD cases (F=8.3; p=0.01) (FIG. 3b). Further analysis revealed that VPS35 and VPS26 levels are correlated with each other (beta=0.67 p=0.007) (FIG. 3b), supporting the established relationship between these proteins.

AD is a slowly progressing disease, and abnormally high Aβ levels observed in the entorhinal cortex and other brain regions of AD patients⁴² likely exists for many years prior to autopsy. It is therefore impossible to rely on autopsy material to determine whether the observed reduction in VPS35 is an upstream defect—causing the elevation in Aβ—or rather a secondary response to neurotoxicity in a dying neuron. To test whether a decrease in VPS35 protein plays a direct role in Aβ production, a series of cell culture experiments were performed in which expression can be experimentally manipulated and Aβ levels measured.

First, siRNA was developed against VPS35 which decreased VPS35 levels by approximately 35% (FIG. 4a), similar to the reductions observed in AD brains (FIG. 3b). When siRNA was introduced into HeLa cells, a significant 37% elevation in endogenous Aβ40 (t=8.2, p=0.001) was observed, as measured with sandwhich ELISA. Since control cases had a relative increase in VPS35 compared to AD brains (FIG. 3b), it was also interesting to determine whether an increase in VPS35 levels could slow APP processing. Also, showing a reverse effect would strengthen the causal link between VPS35 and AP production. Accordingly, vectors expressing VPS35 that increase the concentration of VPS35 protein in stably transfected cells (FIG. 4b) were developed. It was discovered that increasing VPS35 causes a significant 40% reduction in endogenous AP40 levels (t=3.5, p=0.02) (FIG. 4b). Neither VPS35 siRNA nor VPS35 expression vectors significantly effected actin or full length APP.

These results establish that components of the retromer trafficking complex regulate the local levels of Aβ40. Although the interpretation that VPS35 accelerates Aβ production is favored, the possibility that the retromer plays a role in trafficking APP or AP to sites of degradation is not ruled-out. Importantly, by showing that components of the retromer play a role in a molecular pathway relevant AD pathogenesis, these cell culture findings provide a validation of the model-guided microarray results.

TABLE 1
mRNA levels of 33 molecules conformed to the
spatial component of the model

| Name | Genbank Acc. number | P value |
|---|---|---|
| RBP1-like protein | AA887480 | 0.001 |
| Eukaryotic translation factor 2 | AA577698 | 0.001 |
| RARG-1 | NM_016167 | 0.001 |
| Paladin | AU157932 | 0.001 |
| MMAC1 | AF023139 | 0.001 |
| VPS35 | NM_018206 | 0.002 |
| beclin-1 | NM_003766 | 0.002 |
| similar to BRX | AK022014 | 0.002 |
| SPARC-like 1 | NM_004684 | 0.002 |
| Claudin 10 | NM_006984 | 0.002 |
| KIAA0251 | AA643304 | 0.002 |
| cullin 5 | BF435809 | 0.002 |
| COP9 homolog | BC003090 | 0.003 |
| presenilin-associated protein | AF189289 | 0.003 |
| GABA-A receptor-associated protein | AF180519 | 0.003 |
| proteasome beta 4 subunit | NM_002796 | 0.004 |
| MMAC1 | AF023139 | 0.004 |
| FLJ21156 | NM_024602 | 0.004 |
| CDIPT | NM_006319 | 0.004 |
| peroxisomal biogenesis factor 12 | NM_000286 | 0.004 |
| zinc finger protein 262 | NM_005095 | 0.004 |
| Ariadne homolog 2 | NM_006321 | 0.004 |
| Rho guanine exchange factor | AB002380 | 0.004 |
| similar to hypoxia inducible factor3α | AK021881 | 0.004 |
| FLJ12666 | NM_024595 | 0.004 |
| Nucleobindin 2 | NM_005013 | 0.005 |
| aldo-keto reductase 1, C3 | AB018580 | 0.005 |
| FLJ12179 | NM_024662 | 0.005 |
| FLJ22502 | AK026155 | 0.007 |
| succinate-CoA ligase α | AL050226 | 0.007 |
| KIAA0233 | NM_014745 | 0.007 |
| aldehyde dehydrogenase 7, A1 | AU149534 | 0.008 |
| Histone acetyltransferaste (HBOA) | NM_007067 | 0.009 |

Discussion

As generally acknowledged, the experimental power of microarray-the ability to assess thousands of molecules simultaneously-is also its main analytic liability. Addressing the high false-positive rate that naturally occurs with multiple comparisons has emerged as a general problem³⁸. Attempting to solve this problem by acquiring data from thousands of tissue samples is considered impractical; and, since expression levels are not independent events, applying simple statistical corrections is considered inappropriate. A number of analytic approaches are well suited for dealing with type-I error and false-positivity, as employed in other experimental systems where thousands of interconnected variables are generated. For example, statistical techniques like principle components analysis can be used, looking for covariate patterns among groups of variables within a complex dataset⁴³. Alternatively, a complex dataset can be approached with an a priori hypothesis, explicitly searching for a single variable, or a single set of variables, that best matches a prediction. Of course, this model-driven approach is only as good as the hypothesis and any findings require independent validation.

In this study the latter approach was used, first relying on prior histological and imaging studies to generate a model predicting how a molecule associated with AD should behave, then forward-applying this model onto a microarray dataset, and finally using cell culture studies to validate the finding. Using this approach the retromer trafficking molecule, VPS35, whose expression is abnormal in AD tissue and regulates AP levels, was isolated. First described in yeast⁴⁴, the retromer trafficking complex is made up of VPS35, VPS26, and VPS29, and traffics the type-I membrane protein VPS10 from the vacuole back to the trans-golgi-network (TGN)⁴⁵⁻⁴⁷. VPS35 is the molecular core of the retromer, not only binding VPS26 and VPS29, but also acting as the ‘receptor’ for the complex by recognizing and binding VPS10. Reducing the expression of either VPS35 or VPS10 has overlapping effects, leading to mis-trafficking and redistribution of retromer cargo⁴⁵⁻⁴⁷.

Because of the potential importance of its itinerary, the mammalian retromer has been the focus of a growing number of studies⁴⁸. The mammalian orthologs of VPS35, VPS26, VPS29, and VPS10, have been identified and all are expressed in the brain and localize predominately to the endosome⁴⁹⁻⁵¹. In contrast to yeast, mammals express not one but a family of VPS10-containing proteins-including, SorLA, Sortilin, SorCS1, SorCS2, SorCS3, and SORCA⁵. Nevertheless, dysfunction of the mammalian retromer, caused by reducing the levels of VPS26 and VPS35, results in a mis-trafficking and a redistribution of Sortilin to the endosome³⁹, suggesting a conservation of function. Interestingly, the brain is the organ with the highest expression of this family of VPS10-containing proteins⁵¹. SorLA is of ps;ticlar interest, because of its high expression in the entorhinal cortex⁵², because it has a putative APP-binding domain⁵³, and because a prior study have implicated SorLA in AD⁵⁴ Thus, retromer dysfunction might result in mis-trafficking of SorLA and a subsequent increased distribution of APP to the endosome, an organelle in which BACE (β-site APP-cleaving enzyme) activity is maximized⁵⁵. Alternatively, retromer dysfunction may alter the trafficking of APP or Aβ peptide to sites of degradation.

By reducing the levels of VPS26 and VPS35 to induce retromer dysfunction, studies have documented that the mammalian retromer traffics other type-I membrane proteins besides VPS10-containing proteins, such as the mannose-6-phosphate receptor^{39, 40} and the polymeric immunoglobulin receptor⁴¹. A recent study suggests that BACE, a type-I membrane protein with sequence homologies to this group of cargo proteins, might also be trafficked by the mammalian retromer⁵⁶. As reported, a reduction in VPS26, which itself causes a concomitant reduction in VPS35³⁹⁻⁴¹, leads to a mis-trafficking of BACE, increasing its concentrations in the endosome. Here again, whether BACE is trafficked directly by the retromer or indirectly by VPS10-containing receptors remains unknown. In any case, direct or indirect trafficking of BACE or APP by the neuronal retromer, and the redistribution of either molecule when VPS35 is reduced, provides cellular mechanisms that can account for our findings.

More generally, the observed reduction in VPS35 and VPS26, and the fact that this reduction results in elevated Aβ levels, highlights an unexplored cellular pathway that can contribute to the elevated Aβ found in the entorhinal cortex and other brain regions of sporadic AD patients (FIG. 5). The mistrafficking and redistribution of potentia l retromer cargo
SorLA, BACE, or APP-provides a cellular mechanism for alterations in Aβ levels that can, in principle, occur independent of molecular defects in APP, BACE, or components of the γ secretase (FIG. 5).

Why VPS35 is reduced in the first place remains an outstanding question. The fact that mRNA and protein levels of VPS35 are inversely correlated might provide some clues²⁹⁻³², since this relationship suggests either that VPS35 protein undergoes accelerated degradation in the entorhinal cortex of AD patients or that VPS35 mRNA is turner over more slowly.

Isolating the primary molecular defects of autosomal-dominant AD heralded a new era in AD research, and served as the cornerstone upon which insights into the molecular biology of AD have been made. Expressing these molecules in cells and then, ultimately, in genetically engineered mice has resolved many questions about APP processing and the neurotoxic effects of the Aβ peptide. Nevertheless, the molecules defective in autosomal-dominant AD are normal in sporadic AD, the main form of the disease accounting for the vast majority of all cases. Although a complex disorder, isolating the primary molecular defects of sporadic AD is widely acknowledged as a next important step in unraveling the molecular causes of this devastating disease. Even more so than autosomal-dominant AD, numerous molecular defects are expected to contribute to sporadic AD, which in turn, will secondarily affect other molecular pathways. Indeed, a number of microarray studies investigating tissue extracted from AD brains have identified a range of molecular changes^{23, 57-62}. An advantage of microarray is that it interrogates all molecules simultaneously thereby increasing the odds of isolating primary molecular contributors.

Because the spatiotemporal criteria applied to the microarray dataset might have been overly stringent, and because of the limited number of brains investigated, our study does not exclude the importance of other molecular pathways identified in this and in prior microarray studies^{23, 57-62}. Furthermore, although the control brains used for the analysis did not fulfill histological criteria for AD, the possibility that that these brains are free of the earliest pre-symptomatic stages of disease cannot be ruled out, which may not manifest clear histological features. Nevertheless, the strictness of the criteria, and the subsequent validation of the finding in cell culture, lead to the conclusion that a reduction in components of the retromer is as at least one important, and novel, contributor to sporadic AD.

### References

1. Hardy J, Selkoe D J. The amyloid hypothesis of Alzheimer's disease: progress and problems on the road to therapeutics. Science. 2002;297:353-356
2. Price D L, Tanzi R E, Borchelt DR, Sisodia SS. Alzheimer's disease: genetic studies and transgenic models. Annu Rev Genet. 1998;32:461-493
3. Pongrac J, Middleton F A, Lewis D A et al. Gene expression profiling with DNA microarrays: advancing our understanding of psychiatric disorders. Neurochem Res. 2002;27:1049-1063
4. Gu C C, Rao D C, Stormo G et al. Role of gene expression microarray analysis in finding complex disease genes. Genet Epidemiol. 2002;23:37-56
5. Small S A, Pierce A, Kent K et al. Combining functional imaging with microarray; identifying an unexplored cellular pathway implicated in sporadic Alzheimer's disease. The 34th annual meeting of the Society for Neuroscience. New Orleans, 2003
6. Jacobs D M, Sano M, Dooneief G et al. Neuropsychological detection and characterization of preclinical Alzheimer's disease [comment] [see comments]. Neurology. 1995;45:957-962
7. Masur D M, Sliwinski M, Lipton R B et al.

Neuropsychological prediction of dementia and the absence of dementia in healthy elderly persons [see comments]. Neurology. 1994;44:1427-1432
8. Lein E S, Zhao X, Gage F H. Defining a molecular atlas of the hippocampus using DNA microarrays and high-throughput in situ hybridization. J Neurosci. 2004;24:3879-3889
9. Zhao X, Lein E S, He A et al. Transcriptional profiling reveals strict boundaries between hippocampal subregions.

J Comp Neurol. 2001;441:187-196
10. Small S A. Age-related memory decline; current concepts and future directions. Archives of Neurology. 2001;58:360-364
11. Price J L, Ko A I, Wade M J et al. Neuron number in the entorhinal cortex and CA1 in preclinical Alzheimer disease. Arch Neurol. 2001;58:1395-1402
12. Fukutani Y, Cairns N J, Shiozawa M et al. Neuronal loss and neurofibrillary degeneration in the hippocampal cortex in late-onset sporadic Alzheimer's disease. Psychiatry Clin Neurosci. 2000;54:523-529
13. Giannakopoulos P, Herrmann F R, Bussiere T et al. Tangle and neuron numbers, but not amyloid load, predict cognitive status in Alzheimer's disease. Neurology. 2003;60:1495-1500
14. Braak H, Braak E. Evolution of the neuropathology of Alzheimer's disease. Acta Neurol Scand Suppl. 1996;165:3-12
15. Shoghi-Jadid K, Small G W, Agdeppa E D et al. Localization of neurofibrillary tangles and beta-amyloid plaques in the brains of living patients with Alzheimer disease. Am J Geriatr Psychiatry. 2002;10:24-35
16. Schonheit B, Zarski R, Ohm T G. Spatial and temporal relationships between plaques and tangles in Alzheimer-pathology. Neurobiol Aging. 2004;25:697-711
17. Thal D R, Rub U, Orantes M, Braak H. Phases of A beta-deposition in the human brain and its relevance for the development of AD. Neurology. 2002;58:1791-1800
18. West M J, Kawas C H, Stewart W F et al. Hippocampal neurons in pre-clinical Alzheimer's disease. Neurobiol Aging. 2004;25:1205-1212
19. West M J. Regionally specific loss of neurons in the aging human hippocampus. Neurobiol Aging. 1993;14:287-293
20. Simic G, Kostovic I, Winblad B, Bogdanovic N. Volume and number of neurons of the human hippocampal formation in normal aging and Alzheimer's disease. J Comp Neurol. 1997;379:482-494
21. Blalock E M, Geddes I W, Chen K C et al. Incipient Alzheimer's disease: microarray correlation analyses reveal major transcriptional and tumor suppressor responses. Proc Natl Acad Sci USA. 2004;101:2173-2178
22. Colangelo V, Schurr J, Ball M J et al. Gene expression profiling of 12633 genes in Alzheimer hippocampal CA1: transcription and neurotrophic factor down-regulation and up-regulation of apoptotic and pro-inflammatory signaling. J Neurosci Res. 2002;70:462-473
23. Ginsberg S D, Hemby S E, Lee V M et al. Expression profile of transcripts in Alzheimer's disease tangle-bearing CA1 neurons. Ann Neurol. 2000;48:77-87
24. Selkoe D J. Alzheimer's disease is a synaptic failure. Science. 2002;298:789-791
25. Small S, Wu E, Bartsch D et al. Imaging physiologic dysfunction of individual hippocampal subregions in humans and genetically modified mice. Neuron. 2000:653-664
26. Small S A, Nava A S, Perera G M et al. Evaluating the function of hippocampal subregions with high-resolution MRI in Alzheimer's disease and aging [In Process Citation]. Microsc Res Tech. 2000;51:101-108
27. Small S A, Tsai WY, DeLaPaz R et al. Imaging hippocampal function across the human life span: is memory decline normal or not? Ann Neurol. 2002;51:290-295
28. de Leon M J, Convit A, Wolf O T et al. Prediction of cognitive decline in normal elderly subjects with 2-[(18)F]fluoro-2-deoxy-D-glucose/poitron-emission tomography (FDG/PET). Proc Natl Acad Sci USA. 2001;98:10966-10971
29. Beyer A, Hollunder J, Nasheuer H P, Wilhelm T. Post-transcriptional expression regulation in the yeast Saccharomyces cerevisiae on a genomic scale. Mol Cell Proteomics. 2004;3:1083-1092
30. Chen G, Gharib T G, Huang C C et al. Discordant protein and mRNA expression in lung adenocarcinomas. Mol Cell Proteomics. 2002;1:304-313
31. Tian Q, Stepaniants S B, Mao M et al. Integrated genomic and proteomic analyses of gene expression in Mammalian cells. Mol Cell Proteomics. 2004;3:960-969
32. Greenbaum D, Colangelo C, Williams K, Gerstein M. Comparing protein abundance and mRNA expression levels on a genomic scale. Genome Biol. 2003;4:117
33. Ideker T, Thorsson V, Ranish J A et al. Integrated genomic and proteomic analyses of a systematically perturbed metabolic network. Science. 2001;292:929-934
34. Lee P S, Shaw L B, Choe L H et al. Insights into the relation between mrna and protein expression patterns: II. Experimental observations in Escherichia coli. Biotechnol Bioeng. 2003;84:834-841
35. Mehra A, Lee K H, Hatzimanikatis V. Insights into the relation between mRNA and protein expression patterns: I. Theoretical considerations. Biotechnol Bioeng. 2003;84:822-833
36. Elbashir S M, Martinez J, Patkaniowska A et al. Functional anatomy of siRNAs for mediating efficient RNAi in Drosophila melanogaster embryo lysate. Embo J. 2001;20:6877-6888
37. Elbashir S M, Harborth J, Lendeckel W et al. Duplexes of 21-nucleotide RNAs mediate RNA interference in cultured mammalian cells. Nature. 2001;411:494-498
38. Slonim D K. From patterns to pathways: gene expression data analysis comes of age. Nat Genet. 2002;32 Suppl:502-508
39. Seaman M N. Cargo-selective endosomal sorting for retrieval to the Golgi requires retromer. J Cell Biol. 2004;165:111-122
40. Arighi C N, Hartnell L M, Aguilar RC et al. Role of the mammalian retromer in sorting of the cation-independent mannose 6-phosphate receptor. J Cell Biol. 2004;165:123-133
41. Verges M, Luton F, Gruber C et al. The mammalian retromer regulates transcytosis of the polymeric immunoglobulin receptor. Nat Cell Biol. 2004;6:763-769
42. Lue L F, Kuo Y M, Roher A E et al. Soluble amyloid beta peptide concentration as a predictor of synaptic change in Alzheimer's disease. Am J Pathol. 1999;155:853-862
43. Habeck C, Hilton H J, Zarahn E et al. Relation of cognitive reserve and task performance to expression of regional covariance networks in an event-related fMRI study of nonverbal memory. Neuroimage. 2003;20:1723-1733
44. Paravicini G, Horazdovsky B F, Emr S D. Alternative pathways for the sorting of soluble vacuolar proteins in yeast: a vps35 null mutant missorts and secretes only a subset of vacuolar hydrolases. Mol Biol Cell. 1992;3:415-427
45. Nothwehr S F, Ha S A, Bruinsma P. Sorting of yeast membrane proteins into an endosome-to-Golgi pathway involves direct interaction of their cytosolic domains with Vps35p. J Cell Biol. 2000;151:297-310
46. Seaman M N, Marcusson E G, Cereghino J L, Emr S D. Endosome to Golgi retrieval of the vacuolar protein sorting receptor, VpslOp, requires the function of the VPS29, VPS30, and VPS35 gene products. J Cell Biol. 1997;137:79-92
47. Seaman M N, McCaffery J M, Emr S D. A membrane coat complex essential for endosome-to-Golgi retrograde transport in yeast. J Cell Biol. 1998;142:665-681
48. Seaman M N. Recycle your receptors with retromer. Trends Cell Biol. 2005;15:68-75
49. Edgar A J, Polak J M. Human homologues of yeast vacuolar protein sorting 29 and 35. Biochem Biophys Res Commun. 2000;277:622-630
50. Haft C R, de la Luz Sierra M, Bafford R et al. Human orthologs of yeast vacuolar protein sorting proteins Vps26, 29, and 35: assembly into multimeric complexes. Mol Biol Cell. 2000;11:4105-4116
51. Hampe W, Rezgaoui M, Hermans-Borgmeyer I, Schaller H C. The genes for the human VPS10 domain-containing receptors are large and contain many small exons. Hum Genet. 2001; 108:529-536
52. Motoi Y, Aizawa T, Haga S et al. Neuronal localization of a novel mosaic apolipoprotein E receptor, LRll, in rat and human brain. Brain Res. 1999;833:209-215
53. Rebeck G W, Moir R D, Mui S et al. Association of membrane- bound amyloid precursor protein APP with the apolipoprotein E receptor LRP. Brain Res Mol Brain Res. 2001; 87:238-245
54. Scherzer C R, Offe K, Gearing M et al. Loss of apolipoprotein E receptor LR11 in Alzheimer disease. Arch Neurol. 2004;61:1200-1205
55. Vassar R, Bennett B D, Babu-Khan S et al. Beta-secretase cleavage of Alzheimer's amyloid precursor protein by the transmembrane aspartic protease BACE. Science. 1999;286:735-741
56. He X, Li F, Chang W P, Tang J. GGA proteins mediate the recycling pathway of memapsin 2 (BACE). J Biol Chem. 2005
57. Loring J F, Wen X, Lee J M et al. A gene expression profile of Alzheimer's disease. DNA Cell Biol. 2001;20:683-695
58. Pasinetti G M. Use of cDNA microarray in the search for molecular markers involved in the onset of Alzheimer's disease dementia. J Neurosci Res. 2001;65:471-476
59. Ho L, Guo Y, Spielman L et al. Altered expression of a-type but not b-type synapsin isoform in the brain of patients at high risk for Alzheimer's disease assessed by DNA microarray technique. Neurosci Lett. 2001;298:191-194
60. Mufson E J, Counts S E, Ginsberg S D. Gene expression profiles of cholinergic nucleus basalis neurons in Alzheimer's disease. Neurochem Res. 2002;27:1035-1048
61. Yao P J, Zhu M, Pyun E I et al. Defects in expression of genes related to synaptic vesicle trafficking in frontal cortex of Alzheimer's disease. Neurobiol Dis. 2003;12:97-109
62. Walker P R, Smith B, Liu Q Y et al. Data mining of gene expression changes in Alzheimer brain. Artif Intell Med. 2004;31:137-154

1. A method for determining whether an agent causes an increase in the expression of a retromer complex protein, comprising the steps of:
(a) contacting the agent with a eukaryotic cell under conditions which, in the absence of the agent, permit expression of the retromer complex protein;
(b) after a suitable period of time, determining the amount of expression in the cell of the retromer complex protein; and
(c) comparing the amount of expression determined in step (b) with the amount of expression which occurs in the absence of the agent, whereby an increased amount of expression in the presence of the agent indicates that the agent causes an increase in the expression of the retromer complex protein.

2. The method of claim 1, wherein the retromer complex protein is VPS35.

3. The method of claim 1, wherein the retromer complex protein is selected from the group consisting of VPS17, VPS26, VPS29, SorLa, sorting nexin 1 and sorting nexin 2.

4. The method of claim 1, wherein the cell is present in a cell culture.

5. The method of claim 1, wherein the cell is a brain cell.

6. The method of claim 1, wherein determining the amount of expression is performed by determining the amount of retromer complex protein-encoding mRNA in the cell.

7. The method of claim 1, wherein determining the amount of expression is performed by determining the amount of retromer complex protein in the cell.

8. The method of claim 7, wherein determining the amount of retromer complex protein in the cell is performed using an antibody specific for such protein.

9-13. (canceled)

14. A method for increasing the expression of a retromer complex protein in a cell comprising introducing into the cell an agent which specifically increases the expression of the retromer complex protein in the cell.

15. The method of claim 14, wherein the retromer complex protein is VPS35.

16. The method of claim 14, wherein the retromer complex protein is selected from the group consisting of VPS17, VPS26, VPS29, SorLa, sorting nexin 1 and sorting nexin 2.

17. The method of claim 14, wherein the cell is present in a cell culture.

18. The method of claim 14, wherein the cell is a brain cell.

19. The method of claim 14, wherein the agent is a nucleic acid.

20. The method of claim 19, wherein the nucleic acid is an expression vector encoding one or more retromer complex proteins.

21. The method of claim 19, wherein the nucleic acid is an expression vector encoding VPS35.

22. A method for treating a subject afflicted with Alzheimer's disease comprising administering to the subject a therapeutically effective amount of an agent which specifically increases the expression of the retromer complex protein in the cells of the subject's brain which express Aβ peptide.

23. The method of claim 22, wherein the retromer complex protein is VPS35.

24. The method of claim 22, wherein the retromer complex protein is selected from the group consisting of VPS17, VPS26, VPS29, SorLa, sorting nexin 1 and sorting nexin 2.

25. The method of claim 22, wherein the agent is a nucleic acid.

26. The method of claim 25, wherein the nucleic acid is an expression vector encoding one or more retromer complex proteins.

27. The method of claim 25, wherein the nucleic acid is an expression vector encoding VPS35.

28-29. (canceled)

30. A method for determining whether an agent causes an increase in the activity of a retromer complex, comprising the steps of:
(a) contacting the agent with a eukaryotic cell under conditions which, in the absence of the agent, permit activity of the retromer complex;
(b) determining the amount of activity in the cell of the retromer complex; and
(c) comparing the amount of activity determined in step (b) with the amount of activity which occurs in the absence of the agent, whereby an increased amount of activity in the presence of the agent indicates that the agent causes an increase in the activity of the retromer complex.

31. A pharmaceutical composition comprising:
(a) an agent which specifically increases the expression of a retromer complex protein when introduced into a cell; and
(b) a pharmaceutically acceptable carrier.

32. An article of manufacture comprising:
(a) a packaging material having therein an agent which specifically increases the expression of a retromer complex protein when introduced into a cell; and
(b) a label indicting a use for the agent in treating a subject afflicted with Alzheimer's disease.
